(19) United States
(12) Patent Application Publication
(10) Pub. No.: US 2025/0076281 A1
(43) Pub. Date: Mar. 6, 2025

(54) APPARATUS FOR AND METHOD OF MEASURING A SUBSTANCE IN A BREATH SAMPLE

(71) Applicant: ZENAMED LIMITED, Fowlmere (GB)

(72) Inventor: Mark Julian STOCKDALE, Cambridgeshire (GB)

(21) Appl. No.: 18/439,046

(22) Filed: Feb. 12, 2024

(30) Foreign Application Priority Data
Sep. 6, 2023 (GB) 2313584.1
Sep. 25, 2023 (GB) 2314652.5

(51) Int. Cl. G01N 33/497; G16H 10/60; G16H 40/67

(52) U.S. Cl. CPC G01N 33/4972; G16H 10/60; G16H 40/67; G01N 33/4975

(57) ABSTRACT

An apparatus for testing for a substance in a sample of breath. In one example the apparatus includes a blow tube for receiving a sample of breath, a gas sensor for detecting a substance, and a filter positioned between the blow tube and the gas sensor.

### CROSS-REFERENCE TO RELATED APPLICATIONS

The present application claims priority under 35 U.S.C. § 119 and 37 CFR § 1.55 to United Kingdom patent application number 2313584.1 filed Sep. 6, 2023 and titled “APPARATUS FOR AND METHOD OF MEASURING A SUBSTANCE IN A BREATH SAMPLE”, and to United Kingdom patent application number 2314652.5, filed Sep. 25, 2023 and titled “APPARATUS FOR AND METHOD OF MEASURING A SUBSTANCE IN A BREATH SAMPLE”, both of which are hereby incorporated herein by reference in their entireties.

### TECHNICAL FIELD

The disclosure relates to apparatus for and method of measuring a substance in a breath sample.

### BACKGROUND

The realm of substance detection, particularly through breath analysis, has seen considerable progress over the years. Breath analysis is a non-invasive technique extensively employed for detecting a variety of substances, including alcohol and other volatile organic compounds. This technique is grounded on the principle that certain substances, once ingested or inhaled, are absorbed into the bloodstream, and eventually exhaled in the breath. The detection of these substances in the breath can offer valuable insights into an individual's physiological state.

One of the most prevalent applications of breath analysis is in the detection of alcohol consumption. Devices for this purpose, often known as breathalysers, have been in use for decades to determine blood alcohol concentration in suspected cases of drunk driving. These devices typically comprise components for collecting a breath sample, detecting alcohol, and displaying the test results.

In recent times, breath analysis has also found use in healthcare for monitoring the intake of specific medications. For instance, ensuring that patients adhere to their prescribed medication regimen can be challenging. Breath analysis presents a potential solution to this problem by offering a non-invasive and reliable method for monitoring medication intake.

Despite the advancements in breath analysis technology, several challenges persist. For instance, the accuracy and reliability of breath analysis can be influenced by various factors such as the presence of other substances in the breath, the temperature and humidity of the breath sample, and the flow rate of the breath. Various methods have been proposed to overcome these challenges, such as the use of filters to remove unwanted substances, the use of flow sensors to measure the flow rate of the breath, and the use of controlling electronics to process the sensor signals.

Moreover, the design of breath analysis devices can also impact their performance. For instance, the size and shape of the blow tube can affect the collection of the breath sample, the placement of the gas sensor can affect its exposure to the breath sample, and the design of the digital display can affect the readability of the test results. Therefore, there is a need for breath analysis devices that are not only accurate and reliable, but also user-friendly and easy to use. Improvements are desired to overcome shortcomings of existing implementations.

### SUMMARY

In general terms, the present disclosure is directed to an apparatus for testing for a substance in a breath sample, which includes a blow tube to collect the breath sample, a gas sensor to detect the substance, and a hydrophobic filter placed between the blow tube and the gas sensor. Advantageously, the disclosure addresses the challenge of ensuring consistent medication adherence in alcohol dependent individuals by discreetly and reliably measuring a volatile organic compound (VOC) in a patient's breath, thereby confirming the intake of prescribed medication and importantly, abstinence from alcohol.

According to an aspect of the disclosure, there is provided an apparatus for testing for a substance in a sample of breath. The apparatus comprises a blow tube for receiving a sample of breath, a gas sensor for detecting a substance, and a filter positioned between the blow tube and the gas sensor.

According to an aspect of the disclosure, there is provided an apparatus for testing for a substance in a sample of breath. The apparatus comprises a filter, wherein the filter is a hydrophobic filter of 5 micron or less.

Optionally, the apparatus further comprises an inlet at an interface between the blow tube and a housing containing the gas sensor, the inlet having a lesser diameter than the diameter of the blow tube.

Optionally, a filter may be placed in the inlet to stop spital from coming into contact with the hydrophobic filter.

Optionally, the blow tube includes at least one pressure relief port configured to release excess pressure caused by the breath sample entering the inlet.

Optionally, the apparatus further comprises an outlet for the sample of breath.

Optionally, a first pressure sensor is positioned before the inlet and a second pressure sensor positioned after the inlet. Optionally, the first pressure sensor and second pressure sensor are configured to measure absolute pressure. Optionally, the first pressure sensor.

Optionally, the first pressure sensor is positioned before an internal flow restrictor and the second pressure sensor is positioned after the internal flow restrictor.

Optionally, the rate of flow of the breath sample is calculated as a function of a first measurement of absolute pressure measured by the first pressure sensor and a second measurement of absolute pressure measured by the second pressure sensor. In the event that the rate of flow as calculated as a function of the difference in absolute pressure differs by more than a predetermined value, the pump is controlled to vary the flow rate of the sample of breath.

Optionally, the gas sensor is configured to determine the presence of volatile organic compounds in the breath sample.

According to an aspect of the disclosure, there is provided an apparatus for testing for a substance in a sample of breath. The apparatus comprises a gas sensor, which is a photo-ionization detector.

Optionally, the apparatus for testing for a substance in a sample of breath further comprises feedback means configured to indicate a parameter of a breath sample.

According to an aspect of the disclosure, there is provided an apparatus for testing for a substance in a sample of breath. The apparatus comprises feedback means that provides tactile or visual feedback.

Optionally, the apparatus further comprises a wireless communication module configured to submit data concerning analysis of the breath sample to a remote device.

According to an aspect of the disclosure, there is provided an apparatus for testing for a substance in a sample of breath. The apparatus is described above and further comprises a display screen.

According to an aspect of the disclosure, there is provided an apparatus for testing for a substance in a sample of breath. The apparatus further comprises at least one user identification device.

Optionally, the apparatus comprises at least two user identification devices.

Optionally, the substance to be identified is alcohol.

Optionally, the substance to be identified is disulfiram.

According to an aspect of the disclosure, there is provided a method of measuring a substance in a breath sample. The method comprises the steps of: receiving via a handheld device, a breath sample; passing the breath sample through a filter; and determining a presence of a target substance in the filtered breath sample.

According to an aspect of the disclosure, there is provided a method of measuring a substance in a breath sample. The method further comprises the steps of measuring the absolute pressure of the breath sample at a first location, measuring the absolute pressure of the breath sample at a second location, determining that there is a difference in the absolute pressure measured at the second location as compared to that measured at the first location, determining that the difference in absolute pressure is greater than a predetermined value, and controlling a pump via the handheld device to vary the flow rate of the sample of breath.

Optionally, the method further comprises the step of identifying the person providing the breath sample.

Optionally, the method further comprises the step of transmitting data regarding the breath sample to a remote device.

Optionally, the method further comprises the step of providing feedback to the person providing the breath sample to confirm that the breath sample has been correctly processed.

### BRIEF DESCRIPTION OF THE DRAWINGS

The above and other objects and advantages of the disclosure of the disclosures will be apparent upon consideration of the following detailed description, taken in conjunction with the accompanying drawings in which:

FIG. 1 illustratively shows a first embodiment of an apparatus for testing for a substance in a sample of breath according to the present disclosure.

FIG. 2 illustratively shows a second embodiment of an apparatus for testing for a substance in a sample of breath according to the present disclosure.

FIG. 3 illustratively shows a flow control diagram according to the present disclosure.

FIG. 4 illustratively shows a block diagram of apparatus according to the present disclosure.

FIG. 5 illustratively shows a method according to the present disclosure.

### DETAILED DESCRIPTION

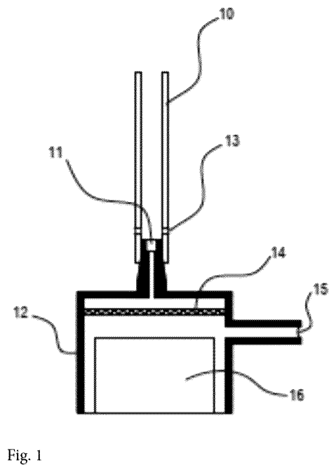

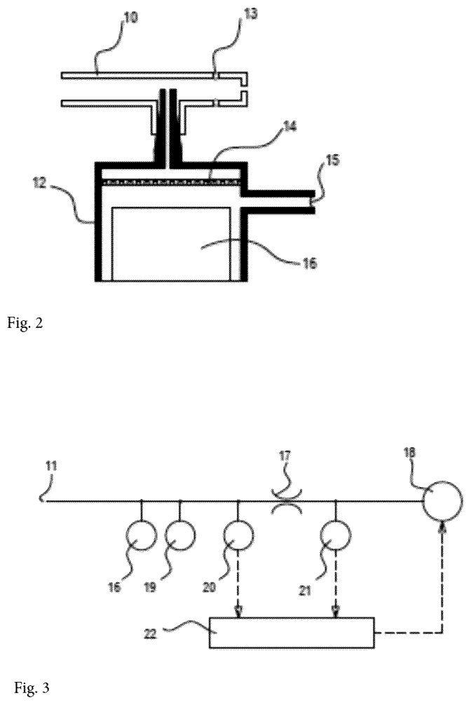

FIGS. 1 and 2 illustratively show embodiments of apparatus for testing for a substance in a sample of breath according to the present disclosure. The apparatus comprises a blow tube (10) for receiving a sample of breath, a gas sensor (16) for detecting the substance, and a hydrophobic filter (14) positioned between the blow tube (10) and the gas sensor (16). In some embodiments the apparatus further comprises an inlet (11) at an interface between the blow tube (10) and a housing (12) containing the gas sensor (16), wherein the inlet (11) has a lesser diameter than the diameter of the blow tube (10). Furthermore, a spital filter (27) may be positioned within the blow tube (10) or inlet (11).

Advantageously, the blow tube (10) serves as the initial point of collection for the breath sample, acting as a conduit for the user's breath to be directed into the apparatus. The blow tube (10) could be ergonomically designed to fit comfortably in the user's mouth, ensuring ease of use and encouraging consistent usage. The user blows into the blow tube (10) providing a sample of their breath for analysis. This breath sample could contain a variety of substances, depending on the user's recent activities and consumption. Importantly, the breath sample is not required to contain alcohol in order for testing of other substances that positively aid sobriety.

The gas sensor (16), which can be a photo-ionization detector (PID) as mentioned in the present disclosure, is responsible for detecting the presence of a specific substance in the breath sample. The PID operates by using ultraviolet light to ionize the molecules in the breath sample. This ionization process causes the molecules to release electrons, which are then measured by the PID to determine the concentration of the target substance. This method of detection is highly sensitive and can accurately detect even minute quantities of the target substance.

The target substance could be a volatile organic compound (VOC) indicative of the user having taken a prescribed medication such as disulfiram. Disulfiram is a medicine commonly used in the treatment of chronic alcoholism, and its presence in the breath sample could indicate that the user is adhering to their prescribed medication regimen. Additionally, or alternatively, the target substance could be alcohol, the consumption of which the user is trying, or is required, to avoid. The presence of alcohol in the breath sample could indicate a relapse, triggering the need for intervention or adjustment of the treatment plan. To identify if alcohol is present in a sample of breath, a secondary sensor (not shown) would be required.

The filter (14), which is positioned between the blow tube (10) and the gas sensor (16), serves to purify the breath sample before it reaches the gas sensor (16). This filter (14) could be made of a variety of materials, such as PTFE. The filter (14) works by trapping and removing moisture from the breath sample that could interfere with the gas sensor's (16) ability to accurately detect the target substance. This ensures that the gas sensor (16) is not overwhelmed by extraneous particles and can accurately detect the target substance.

The filter (14) may be a hydrophobic filter of 5 micron or less. Advantageously, a hydrophobic filter (14) of 5 micron or less serves to remove moisture and other particulates from the breath sample. This is crucial as these elements could potentially interfere with the accurate detection of the target substance by the gas sensor (16). By ensuring that the breath sample is as pure as possible, the hydrophobic filter enhances the reliability of the results obtained from the gas sensor (16).

Advantageously, the filter (14) serves to stop spital and other particles that might block the small gas channels leading to the gas sensor (16), and other sensors. This is important as it keeps the gas pathways clear from becoming blocked and it being located on the outside of the instrument then it is easily replaceable in direct contrast to the considerable difficulty in the cleaning of small internal gas channels.

In some embodiments the blow tube (10) further comprises at least one pressure relief port (13) configured to release pressure in the blow tube (10) caused by the flow of the breath sample being restricted entering the inlet (11) and/or by a separate flow restrictor (17) and resulting in back pressure within the blow tube (10).

Advantageously, the at least one pressure relief port (13) serves to maintain a stable pressure within the blow tube (10). Moreover, positioning of the pressure relief port (13) near the inlet (11) ensures the blow tube volume is quickly swept with 100% breath sample and therefore provides a rapid and reliable measurement response time. By releasing excess pressure, then at least one pressure relief port (13) ensures ease of blowing for the user and maintains a more regular pressure at the inlet (11) for better instrument sampling.

The apparatus may include an outlet (15) from the housing (12). Advantageously, the outlet (15) serves to expel the breath sample after it has been analysed by the gas sensor (16). This ensures that the apparatus is ready to receive a new breath sample for subsequent testing. By expelling the analysed breath sample, an accumulation of breath samples is avoided within the apparatus, which could potentially interfere with the accurate detection of the target substance in future tests. Moreover, the outlet (15) becomes an inlet (not shown) to a second sensor that can directly detect alcohol to determine if the user is trying to circumvent the primary gas sensor's (16) operation.

As shown in FIG. 3, the apparatus may comprise a first pressure sensor (20) that measures the absolute pressure of the breath sample at a first location and a second pressure sensor (21) that measures the absolute pressure of the breath sample at a second location. In the event that the absolute pressure measured by the second pressure sensor (21) differs from the absolute pressure measured by the first pressure sensor (20) by less than a threshold amount, the pump (18) is activated to cause the breath sample to flow at an increased flow rate.

Advantageously, the pump (18) serves to vary the flow rate of the breath sample in the event that the absolute pressure measured by the second flow sensor (21) is significantly different from the absolute pressure measured by the first flow sensor (20). This ensures that the rate of flow of the breath sample is consistent as it flows through the apparatus from the inlet (11) to the outlet (15). By activating the pump (18), the apparatus can adapt to changes in the flow rate of the breath sample seen as varying pressure levels at the blow tube (10). The first pressure sensor (20) may be positioned before a flow restrictor (17) and the second pressure sensor (21) may be positioned after the flow restrictor (17). Furthermore, this feature helps to ensure the final sample entering the detector(s) is more consistent to that presented during the initial calibration of the instrument, unlike that with existing alcohol-only instruments that often use a single pressure increase sensor and rely upon the person presenting the sample to give a (variable) sample volume of gas to the sensor(s) within the instrument.

In addition to the gas sensor (16), the apparatus may comprise additional sensors (19) such as a moisture sensor, pH sensor, temperature sensor, alcohol sensor for example.

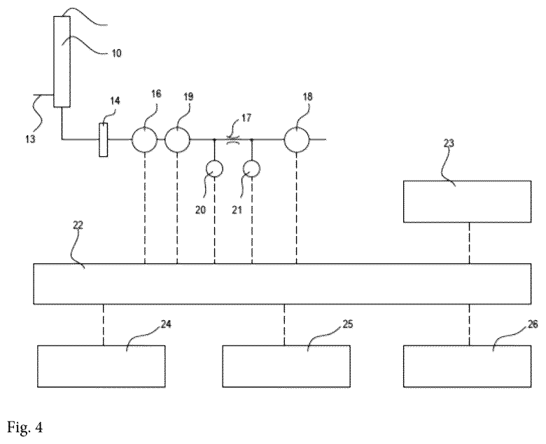

As shown in FIG. 4, once the breath sample has been exposed to the gas sensor (16) and other sensors (19) it is passed to feedback control electronics (22) of the apparatus. The feedback control electronics are operable to control a mechanical interface (24), optical interface (25), and wire interface (26).

In some embodiments the apparatus is configured to determine the presence of volatile organic compounds in the breath sample.

Advantageously, the ability of the apparatus to detect volatile organic compounds (VOCs) in the breath sample is crucial in monitoring the intake of prescribed medication such as disulfiram by patients suffering from alcoholism. The presence of certain VOCs in the breath sample can indicate that the patient is taking the prescribed medication, thereby discouraging the consumption of alcohol. This is because the metabolism of disulfiram in the body produces a VOC that can be detected in the patient's breath. By consistently measuring this VOC, the apparatus can provide a reliable indication of the patient's adherence to the prescribed medication regimen.

In some embodiments the apparatus for testing for a substance in a sample of breath further comprises feedback means configured to indicate a parameter of a breath sample.

Advantageously, the feedback means provides the user with real-time information about the breath sample, such as its flow rate or the concentration of the target substance. This can be particularly useful in ensuring that the user is providing a sufficient breath sample for analysis, and in providing immediate feedback about the presence of the target substance.

The apparatus may include feedback means which provides tactile or visual feedback. Advantageously, the tactile or visual feedback provided by the feedback means enhances the user experience by providing immediate and intuitive information about the breath sample. For example, a visual indicator could change colour to indicate the presence of the target substance, or a tactile indicator could vibrate to signal that a sufficient breath sample has been collected.

In some embodiments the apparatus further comprises a wireless communication module (23) configured to submit data concerning analysis of the breath sample to a remote device.

Advantageously, the wireless communication module (23) allows for the remote monitoring of the user's adherence to the prescribed medication regimen. By transmitting data about the breath sample to a remote device, such as a physician's computer, the apparatus allows for the continuous monitoring of the user's condition, thereby enhancing the effectiveness of the treatment.

The apparatus may comprise a display screen, which is not shown in the figures. Advantageously, the display screen provides a user-friendly interface for the apparatus, allowing the user to easily understand the results of the breath sample analysis. The display screen could show information such as the concentration of the target substance in the breath sample, or it could provide instructions for the user on how to properly use the apparatus.

The apparatus may comprise at least one user identification device, which is not shown in the figures. Advantageously, the user identification device ensures that the breath sample is associated with the correct user. This is particularly important in a clinical setting, where multiple users may be using the apparatus. By accurately identifying the user, the apparatus can provide personalized feedback and data, enhancing the effectiveness of the treatment.

In some embodiments the apparatus for testing for a substance in a sample of breath comprises at least two user identification devices.

Advantageously, the inclusion of at least two user identification devices in the apparatus ensures the authenticity of the breath sample being tested. This is crucial in scenarios where the apparatus is used for monitoring the intake of prescribed medication such as disulfiram by alcohol dependent individuals. By verifying the identity of the user, the apparatus can ensure that the breath sample being tested is indeed from the intended user and not from a different individual. This prevents any potential misuse of the apparatus and ensures the reliability of the results obtained.

User identification devices may include: a keypad, fingerprint scanner, retinal scanner, facial feature scan, for example.

The substance to be identified may be disulfiram. Advantageously, the apparatus may be specifically designed to detect the presence of disulfiram in a breath sample. This is crucial in the treatment of alcoholism, where disulfiram is often prescribed to deter the patient from consuming alcohol. The blow tube (10) serves as the initial point of collection for the breath sample, while the gas sensor (16), which could be a photo-ionization detector (PID) as mentioned in the present disclosure, is responsible for detecting the presence of disulfiram in the breath sample. The filter (14), which is positioned between the blow tube and the gas sensor (16), serves to purify the breath sample before it reaches the gas sensor (16). This ensures that the gas sensor (16) can accurately detect the presence of disulfiram in the breath sample.

The substance to be identified may be alcohol. The apparatus includes features such as sensors, processors, and other components that are configured to detect the presence of alcohol in the sample of breath. Advantageously, the apparatus may be specifically designed to detect the presence of alcohol in a breath sample. This is particularly useful in the treatment of alcoholism, where it is important to monitor the alcohol consumption of the patient. The apparatus includes a variety of components such as sensors and processors that work together to accurately detect the presence of alcohol in the breath sample. The sensors, which could include an electrochemical cell and be responsible for detecting the presence of alcohol in the breath sample. The processors then analyze the data obtained from the sensors to determine the alcohol content of the breath sample.

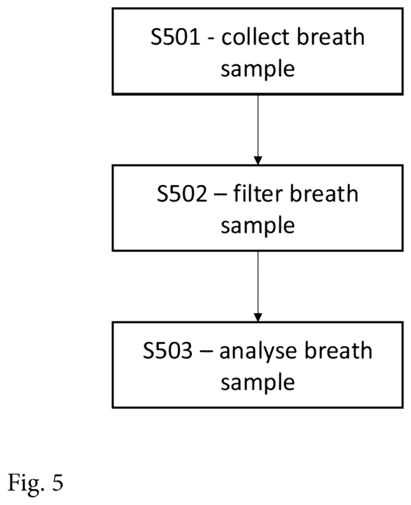

FIG. 5 illustratively shows a flowchart for performing a method according to the present disclosure. The method includes three steps: (S501) receiving a breath sample via a handheld device (10); (S502) passing the breath sample through a filter (14); and (S503) determining the presence of a target substance in the filtered breath sample.

Advantageously, the method shown in FIG. 5 provides a simple and efficient way to test for the presence of a target substance in a breath sample. The breath sample is first received via a handheld device (10), which could be the apparatus described in the present disclosure. The breath sample is then passed through a filter (14), which serves to remove moisture and any extraneous particles to purify the breath sample. Finally, the presence of the target substance in the filtered breath sample is determined. This could be done using a gas sensor (16) such as a photo-ionization detector (PID), which is capable of accurately detecting the presence of the target substance in the breath sample.

The method may include measuring an absolute pressure of the breath sample at a first location (20), measuring the absolute pressure of the breath sample at a second location, determining that there is a pressure drop between the measurement taken at the first location and the measurement taken at the second location, and controlling a pump (18) via the handheld device to vary the flow rate. In the event that the pressure drop is determined to be less than a prescribed value, the pump (18) speed is increased. In the event that the pressure drop is determined to be more than a prescribed value, the pump (18) speed is decreased.

In some embodiments the method further comprises the step of identifying the person providing the breath sample. Advantageously, the step of identifying the person providing the breath sample ensures the authenticity of the breath sample being tested. This is crucial in scenarios where the apparatus is used for monitoring the intake of prescribed medication such as disulfiram by alcohol dependent individuals. By verifying the identity of the user, the apparatus can ensure that the breath sample being tested is indeed from the intended user and not from a different individual. This prevents any potential misuse of the apparatus and ensures the reliability of the results obtained.

In some embodiments the method further comprises the step of transmitting data regarding the breath sample to a remote device. Advantageously, the step of transmitting data regarding the breath sample to a remote device allows for the remote monitoring of the user's intake of the target substance. This could be particularly useful in the treatment of alcoholism, where it is important to monitor the patient's alcohol consumption and intake of prescribed medication such as disulfiram. The data could be transmitted to a device operated by a physician, allowing the physician to monitor the patient's progress and adjust the treatment plan as necessary.

In some embodiments the method further comprises the step of providing feedback to the person providing the breath sample to confirm that the breath sample has been correctly processed. Advantageously, providing feedback to the person providing the breath sample serves to ensure that the sample has been correctly processed. This feedback can be delivered via an optical or wireless interface, as mentioned in the present disclosure. This feedback mechanism is crucial in maintaining the reliability of the apparatus and the accuracy of the results. It allows the user to know that their breath sample has been correctly processed and that the results are accurate. This feedback can also serve to reassure the user that the apparatus is functioning correctly and that their breath sample has been successfully analysed. This can be particularly important in cases where the apparatus is being used by a physician or by the person taking treatment themselves, as it allows them to confidently rely on the results provided by the apparatus.

In some embodiments the method of measuring a substance in a breath sample is used to identify disulfiram and/or alcohol. Advantageously, the method of measuring a substance in a breath sample can be used to identify disulfiram and/or alcohol. This is particularly useful in the treatment of alcoholism, as it allows for the monitoring of the patient's adherence to their prescribed medication regimen that suppresses the patients' desire to consume alcohol. As mentioned in the present disclosure, it is well documented that alcohol dependent individuals who are prescribed and taking disulfiram will not drink alcohol as it makes them violently unwell. However, alcoholism is an illness and sufferers will often avoid taking their medication in order to consume alcohol. By using this method to consistently measure the presence of disulfiram and/or alcohol in the patient's breath, it can be ensured that the patient is taking their prescribed medication and is not consuming alcohol. This can be crucial in the successful treatment of alcoholism, as it allows for the monitoring of the patient's adherence to their medication regimen and their avoidance of alcohol. The results of these measurements can be sent discreetly via an optical or wireless interface for onward analysis by the physician, allowing for the ongoing monitoring of the patient's condition.

It will be understood that the term disulfiram as used herein may refer to a specific medicine commonly used in the treatment of chronic alcoholism, which inhibits an enzyme involved in metabolizing alcohol and produces unpleasant side effects when alcohol is consumed.

It will be understood that the term photo-ionization detector as used herein may refer to a type of gas detector that utilizes ultraviolet light to ionize a gas sample and subsequently measure the current to detect specific volatile organic compounds or other gases in the breath sample.

It will be understood that the term hydrophobic filter (14) as used herein may refer to a filter designed to absorb moisture from the breath sample proceeding to the gas sensor (16) and affecting the gas sensor's sensitivity to disulfiram.

The foregoing disclosure provides illustration and description but is not intended to be exhaustive or to limit the implementations to the precise form disclosed. Modifications may be made considering the above disclosure or may be acquired from practice of the implementations. As used herein, the term “component” is intended to be broadly construed as hardware, firmware, or a combination of hardware and software. It will be apparent that systems and/or methods described herein may be implemented in different forms of hardware, firmware, and/or a combination of hardware and software. The actual specialized control hardware or software code used to implement these systems and/or methods is not limiting of the implementations. Thus, the operation and behavior of the systems and/or methods are described herein without reference to specific software code-it being understood that software and hardware can be used to implement the systems and/or methods based on the description herein. As used herein, satisfying a threshold may, depending on the context, refer to a value being greater than the threshold, greater than or equal to the threshold, less than the threshold, less than or equal to the threshold, equal to the threshold, and/or the like, depending on the context. Although combinations of features are recited in the claims and/or disclosed in the specification, these combinations are not intended to limit the disclosure of various implementations. In fact, many of these features may be combined in ways not specifically recited in the claims and/or disclosed in the specification.

Although each dependent claim listed below may directly depend on only one claim, the disclosure of various implementations includes each dependent claim in combination with every other claim in the claim set. No element, act, or instruction used herein should be construed as critical or essential unless explicitly described as such. Also, as used herein, the articles “a” and “an” are intended to include one or more items and may be used interchangeably with “one or more.” Further, as used herein, the article “the” is intended to include one or more items referenced in connection with the article “the” and may be used interchangeably with “the one or more.” Furthermore, as used herein, the term “set” is intended to include one or more items (e.g., related items, unrelated items, a combination of related and unrelated items, and/or the like), and may be used interchangeably with “one or more.” Where only one item is intended, the phrase “only one” or similar language is used. Also, as used herein, the terms “has,” “have,” “having,” or the like are intended to be open-ended terms. Further, the phrase “based on” is intended to mean “based, at least in part, on” unless explicitly stated otherwise. Also, as used herein, the term “or” is intended to be inclusive when used in a series and may be used interchangeably with “and/or,” unless explicitly stated otherwise (e.g., if used in combination with “either” or “only one of”).

1. An apparatus for testing for a substance in a sample of breath comprising a blow tube for receiving a sample of breath, a gas sensor for detecting a substance, and a filter positioned between the blow tube and the gas sensor.

2. The apparatus for testing for a substance in a sample of breath according to claim 1, wherein the filter comprises a hydrophobic filter of 5 micron or less.

3. The apparatus for testing for a substance in a sample of breath according to claim 1, further comprising an inlet at an interface between the blow tube and a housing containing the gas sensor, the inlet having a lesser diameter than the diameter of the blow tube.

4. The apparatus for testing for a substance in a sample of breath according to claim 1, wherein the blow tube further comprises at least one pressure relief port configured to release pressure in the blow tube caused by the sample of breath entering the inlet.

5. The apparatus for testing for a substance in a sample of breath according to claim 1, further comprising:
a first pressure sensor;
a second pressure sensor; and
a flow restrictor, wherein the first pressure sensor is positioned before the flow restrictor, and the second pressure sensor positioned after the flow restrictor.

6. The apparatus for testing for a substance in a sample of breath according to claim 1, wherein a rate of flow of the sample of breath is calculated as a function of a first measurement of absolute pressure measured by the first pressure sensor and a second measurement of absolute pressure measured by the second pressure sensor.

7. The apparatus for testing for a substance in a sample of breath according to claim 6, further comprising a pump, wherein:
the rate of flow of the sample of breath is calculated as a function of a difference in the first measurement of absolute pressure and the second measurement of absolute pressure that differs by more than a predetermined value; and
the pump is controlled to vary the rate of flow of the sample of breath.

8. The apparatus for testing for a substance in a sample of breath according to claim 1, wherein the gas sensor is configured to determine a presence of a volatile organic compound in the sample of breath.

9. The apparatus for testing for a substance in a sample of breath according to claim 1, wherein the gas sensor comprises a photo-ionization detector.

10. The apparatus for testing for a substance in a sample of breath according to claim 1 further comprising an outlet for the sample of breath.

11. The apparatus for testing for a substance in a sample of breath according to claim 1 further comprising a feedback means configured to indicate a parameter of the sample of breath.

12. The apparatus for testing for a substance in a sample of breath according to claim 11, wherein the feedback means provides a tactile feedback or a visual feedback.

13. The apparatus for testing for a substance in a sample of breath according to claim 1 further comprising a wireless communication module configured to submit data concerning an analysis of the sample of breath to a remote device.

14. The apparatus for testing for a substance in a sample of breath according to claim 1 further comprising a display screen.

15. The apparatus for testing for a substance in a sample of breath according to claim 1 further comprising at least one user identification device.

16. The apparatus for testing for a substance in a sample of breath according to claim 15, wherein the at least one user identification device comprises at least two user identification devices.

17. The apparatus for testing for a substance in a sample of breath according to claim 1, wherein the substance to be identified comprises an alcohol.

18. The apparatus for testing for a substance in a sample of breath according to claim 1, wherein the substance to be identified comprises a disulfiram.

19. A method of measuring a substance in a sample of breath, the method comprising:
receiving, via a handheld device, a sample of breath;
passing the sample of breath through a filter; and
determining a presence of the substance in the filtered sample of breath.

20. The method of measuring a substance in a sample of breath, according to claim 19, the method further comprising the steps of:
measuring an absolute pressure of the sample of breath at a first location;
measuring an absolute pressure of the sample of breath at a second location;
determining a difference in the absolute pressure measured at the second location compared to the absolute pressure measured at the first location;
determining that the difference in absolute pressure is outside a predetermined value; and
controlling a pump via the handheld device to vary a flow rate of the sample of breath.

21. The method of measuring a substance in a sample of breath according to claim 19, further comprising identifying a person providing the sample of breath.

22. The method of measuring a substance in a sample of breath according to claim 19, further comprising transmitting data regarding the sample of breath to a remote device.

23. The method of measuring a substance in a sample of breath according to claim 19, further comprising providing feedback to the person providing the sample of breath to confirm that the breath sample has been correctly processed.

24. The method of measuring a substance in a breath sample according to claim 19, wherein the substance comprises at least one of an alcohol or a disulfiram.
